(19) United States
(12) Patent Application Publication
(10) Pub. No.: US 2002/0068271 A1
(43) Pub. Date: Jun. 6, 2002

(54) Methods for identifying agents capable of modulating protein kinase C theta (PKC0) activity

(76) Inventors: Amnon Altman, La Jolla, CA (US); Nolwenn Coudronniere, San Diego, CA (US)

Correspondence Address: Robert M. Bedgood, Ph.D., PILLSBURY WINTHROP LLP, 50 Fremont Street, San Francisco, CA, 94105, US

(21) Appl. No.: 09/749,956

(22) Filed: Dec. 27, 2000

Related U.S. Application Data
(60) Provisional application No. 60/173,171, filed on Dec. 27, 1999.

(51) Int. Cl. C12Q 1/70; G01N 33/53; C07K 1/00; C07K 17/00; C12Q 1/68; C07H 21/04; C07K 14/00

(52) U.S. Cl. 435/5; 435/6; 435/7.1; 530/350; 536/23.72; 536/23.2

(57) ABSTRACT

The invention provides methods for identifying agents that modulate activities of protein kinase C theta (PKCθ) polypeptides. The invention provides methods for identifying a therapeutic agent for ameliorating an HIV infection. The invention provides methods for ameliorating a condition in a subject (e.g., an HIV infection, a skeletal muscle disorder, an immune disorder) by modulating PKCθ polypeptide activity. The invention also provides for ablation of the CD28 costimulatory signal in T cells, abolishing of a T cell survival signal, and promote the apoptosis of activated self-reactive T cells, e.g., in autoimmune diseases.

### RELATED APPLICATIONS

This application incorporates by reference and claims the benefit of priority under 35 U.S.C. §119(e) of U.S. Provisional Application No. 60/173,171, filed Dec. 27, 1999. The aforementioned application is explicitly incorporated herein by reference in its entirety and for all purposes.

### STATEMENT AS TO FEDERALLY SPONSORED RESEARCH

This invention was made in part with Government support under a grant from National Institutes of Health, CA35299. The Government may have certain rights in the invention.

### TECHNICAL FIELD

This invention generally relates to immunology and medicine. In particular, this invention provides methods for identifying agents that modulate activities of protein kinase C theta (PKCθ) polypeptides. The invention provides methods for identifying a therapeutic agent for ameliorating an HIV infection. The invention provides methods for ameliorating a condition in a subject (e.g., an HIV infection, a skeletal muscle disorder, an immune disorder) by modulating PKCθ polypeptide activity. The invention also provides for ablation of the CD28 costimulatory signal in T cells, abolishing of a T cell survival signal, and promote the apoptosis of activated self-reactive T cells, e.g., in autoimmune diseases.

### BACKGROUND

T cell activation induced by triggering of the antigen-specific T cell receptor (TCR)/CD3 complex in concert with costimulatory and adhesion receptors is a complex process that involves multiple enzymes, including members of the protein kinase C (PKC) family, adapters and other cellular proteins. Activation is initiated by stimulation of TCR-coupled protein tyrosine kinases of the Src and Syk families, which then phosphorylate various cellular substrates. This is followed by the recruitment and assembly of membrane signaling complexes that mediate different signal transduction pathways. These signals are relayed to the nucleus, where they induce a defined genetic program.

Protein kinase C-θ (PKCθ) is a Ca²⁺-independent member, or isoform, of the protein kinase C (PKC) family that is selectively expressed in skeletal muscle and T lymphocytes. PKCθ plays an important role in T cell activation. This is based on the findings that PKCθ specifically activates c-Jun N-terminal kinase (JNK) and AP-1 in T lymphocytes, and synergizes with calcineurin to activate the IL-2 gene (see, e.g., Baier-Bitterlich (1996) Mol. Cell. Biol. 16:1842-1850; Werlen (1998) EMBO J. 17:3101-3111; Ghaffari-Tabrizi (1999) Eur. J. Immunol. 29:132-142). It is also based on finding that PKCθ selectively co-localizes with the T cell antigen receptor (TCR) to the core of the supramolecular activation complex (SMAC) formed in the contact region between antigen-specific T cells and antigen-presenting cells (see, e.g., Monks (1997) Nature 385:83-86; Monks (1998) Nature 395:82-86). However, the molecular basis for these important functions of PKCθ in T cells, and the manner in which it becomes coupled to the TCR signaling machinery, are unknown.

### SUMMARY

The invention provides a method for identifying an agent that modulates an activity of a PKCθ polypeptide comprising: (a) providing a PKCθ polypeptide and a test agent; (b) contacting the PKCθ polypeptide with the test agent; and, (c) determining the activity of the PKCθ polypeptide, wherein an increase or decrease in activity of the PKCθ polypeptide in the presence of the test agent thereby identifies the agent as a modulator of PKCθ polypeptide activity. In the methods of the invention the PKCθ polypeptide can be any functional analog, mimetic or variant. The PKCθ polypeptide can be derived from any source, including mammalian, e.g., human. For example, the PKCθ comprises a polypeptide encoded by a nucleic acid comprising a sequence as set forth in SEQ ID NO:1 or comprising an amino acid sequence as set forth in SEQ ID NO:2.

In one aspect of the methods, the PKCθ polypeptide is provided by recombinant expression of a nucleic acid encoding a PKCθ polypeptide. The nucleic acid encoding a PKCθ polypeptide can be in the form of a cDNA or a genomic fragment comprising a PKCθ coding sequence. The nucleic acid can comprise a sequence as set forth in SEQ ID NO:1 or a nucleic acid encoding an amino acid sequence as set forth in SEQ ID NO:2. The nucleic acid can be recombinantly expressed in vitro or in vivo. The nucleic acid can be in the form of an expression cassette, e.g., an expression vector, a recombinant virus, as a stably incorporated gene, e.g., as in a non-human transgenic animal model. The nucleic acid can be expressed in a transfected or infected cell. The cell can be transiently or stably transfected. The in vivo expression can comprise expression of a heterologous PKCθ polypeptide in a non-human transgenic animal.

In alternative aspect of the methods of the invention, the activity of the PKCθ polypeptide is determined by measuring the activity of an IβB-kinase β (IKKβ) or by measuring the activity of an NFκB. In one aspect, the activity of the PKCθ polypeptide is determined using a phosphorylation assay, e.g., the phosphorylation of an endogenous or an exogenous substrate is measured. The substrate can be directly phosphorylated by a PKCθ, or, alternatively, the substrate can be phosphorylated by a kinase directly or indirectly activated or suppressed by PKCθ.

In another aspect of the invention, the activity of the PKCθ polypeptide is determined by measuring the activity of a reporter construct, e.g., a nucleic acid (such as an expression vector) comprising an inducible transcriptional regulatory element (e.g., a promoter, a cis-acting “motif”) and a coding sequence for a detectable polypeptide or an enzyme (e.g., luciferase). For example, the inducible promoter can comprise an NFκB-responsive element, such as a viral promoter, e.g., an HIV-1 promoter. Alternatively, the NFκB-responsive element can comprise an IL-2 promoter or a CD28RE/AP-1 element. Thus, in one aspect, the activity of the PKCθ polypeptide is determined by detecting a change in a promoter's (e.g., an HIV promoter) activity. In alternative aspects, the reporter construct can comprise an AP-1 element, a fas-ligand promoter, or an SRE or “serum responsive element” promoter.

In one aspect, the PKCθ polypeptide and a reporter construct (encoding a detectable polypeptide) are co-expressed, e.g., they are co-transfected and co-expressed in a cell assay system. The agent to be tested is contacted with the cell before, during or after expression of the recombinant polypeptides. Alternatively, the PKCθ polypeptide can be co-expressed with a substrate for the PKCθ kinase, or, a substrate for a kinase activated or inhibited by the PKCθ kinase, e.g., an IβB-kinase β (IKKβ).

In alternative aspects, the agent can modulate (i.e., inhibit or stimulate) an activity of the PKCθ polypeptide, e.g., a phosphorylation event, or detecting binding of the PKCθ (e.g., to another polypeptide, such as an element in the TCR “synapse,” also called the supramolecular activation complex (SMAC) or the immunological synapse), or a PKCθ kinase activity, or another “downstream” PKCθ kinase event. For example, the agent can inhibit an IβB-kinase β (IKKβ) activity or an NFκB activity (e.g., activation of an IL-2 or an HIV promoter), including detecting activation of NFκB or an NFκB signaling pathway.

In one aspect, determining an activity of the PKCθ polypeptide comprises detecting the ability of the test compound to inhibit the translocation of a PKCθ polypeptide to a cell membrane after co-stimulation of a T cell receptor (TCR) and a CD28. For example, the test compound can inhibit the translocation of a PKCθ polypeptide to a T cell synapse in the cell membrane. The test compound can also inhibit the interaction of or binding of a PKCθ polypeptide to a cytoskeletal protein or a cytoskeleton-associated protein or lipid rafts in the cell membrane. In one aspect of the method, determining an activity of the PKCθ comprises detecting a tyrosine kinase p59fyn activity.

The invention provides methods for identifying a therapeutic agent for ameliorating an HIV infection comprising: (a) providing a PKCθ polypeptide and a test agent; (b) contacting the PKCθ polypeptide with the test agent; and, (c) determining the activity of the PKCθ polypeptide, wherein an increase or decrease in activity of the PKCθ polypeptide in the presence of the test agent thereby identifies the agent as a modulator of PKCθ polypeptide activity and a therapeutic agent for ameliorating an HIV infection. The invention also provides methods for identifying a therapeutic agent for ameliorating an HIV infection comprising: (a) recombinantly expressing a PKCθ polypeptide in a cell; (b) contacting the cell with a test agent; and, (c) determining the activity of the PKCθ polypeptide, wherein an increase or decrease in activity of the PKCθ polypeptide in the presence of the test agent thereby identifies the agent as a modulator of PKCθ polypeptide activity and a therapeutic agent for ameliorating an HIV infection. In one aspect, the activity of the PKCθ polypeptide can be determined by measuring the activity of a reporter construct, which can be an inducible transcriptional regulatory element (e.g., a promoter) operably linked to a detectable protein or an enzyme (e.g., luciferase). The reporter construct can comprise an IL-2 or an HIV-1 promoter. Detecting an activity of PKCθ can comprise detecting a change in the promoter's (e.g., HIV promoter's) activity.

The invention provides a method for ameliorating a condition in a subject, wherein the condition can be ameliorated by modulating PKCθ polypeptide activity, comprising administering to a subject a pharmaceutical formulation comprising an effective amount of an agent capable of modulating a PKCθ polypeptide activity, thereby ameliorating the condition in the subject. For example, the invention provides a method for ameliorating an HIV infection in a subject, comprising administering to a subject a pharmaceutical formulation comprising an effective amount of an agent capable of modulating a PKCθ polypeptide activity, thereby ameliorating the HIV infection in the subject. The invention provides a method for ameliorating an immune disorder in a subject, comprising administering to a subject a pharmaceutical formulation comprising an effective amount of an agent capable of modulating a PKCθ polypeptide activity, thereby ameliorating the immune disorder (e.g., a graft versus host disease, autoimmune disease) in the subject. The invention provides a method for ameliorating a skeletal muscle disorder in a subject, comprising administering to a subject a pharmaceutical formulation comprising an effective amount of an agent capable of modulating a PKCθ polypeptide activity, thereby ameliorating the skeletal muscle disorder in the subject.

The invention provides methods for ameliorating a condition in a subject, wherein the condition can be ameliorated by modulating PKCθ polypeptide activity, comprising administering to a subject a pharmaceutical formulation comprising an effective amount of an agent capable of modulating a PKCθ polypeptide activity, thereby ameliorating the condition in the subject, wherein the agent is a dominant negative or dominant positive PKCθ polypeptide, a nucleic acid comprising a PKCθ antisense sequence capable of modulating the expression of a PKCθ polypeptide in a cell, a PKCθ-specific antibody, a rottlerin or a functional equivalent thereof, a composition that inhibits binding of a lipid cofactor to PKCθ, or a composition that binds a nucleic acid regulating PKCθ expression.

The methods of the invention also provide for ablation of the CD28 costimulatory signal in T cells, abolishing of a T cell survival signal, and promote the apoptosis of activated self-reactive T cells, e.g., in autoimmune diseases.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1 is a schematic of data from a studies demonstrating that CD28 costimulation enhances membrane translocation and in situ catalytic activity of PKCθ, as described in detail in Example 1, below. FIG. 1a: Jurkat T cells were stimulated with anti-CD3 and/or anti-CD28 antibodies (2 μg/ml each) for the indicated times (0, 1, 10, or 30 minutes). Whole extract, cytosol and membrane fractions were prepared, resolved by SDS-PAGE, and the expression of PKCθ in each fraction was determined by Western blotting. PMA stimulation (100 ng/ml for 10 min) was used as a positive control for PKC translocation. The position of PKCθ is indicated by arrowheads. FIG. 1b: Jurkat cells were stimulated as in 1a. Endogenous PKCθ was immunoprecipitated and its enzymatic activity was determined (top panel, “MBP” is substrate for PKCθ). Kinase reactions were performed in the absence of lipid cofactors or PMA to reflect the in situ activity of PKCθ. SDS-PAGE-resolved reaction products were analyzed by autoradiography (top panel). The membrane was immunoblotted with a PKCθ-specific antibody (bottom panel).

FIG. 2 is a schematic, and summary, of data from studies demonstrating that PKCθ selectively activates NF-κB and the CD28RE/AP-1 element of the IL-2 promoter in a T cell-specific manner, as described in detail in Example 1, below. One×10⁷ Jurkat T cells (a, b) or 2×10⁶ 293T cells (c) were transfected with CD28RE/AP-1 (a) or NF-κB-Luc (b, c) reporters (5 μg each) in the presence of empty vector (pEF; -) or constitutively active (A/E) PKC-θ, -α, -ζ or -ε mutants (10 μg each). After 24 hr, cells were lysed and luciferase activity was quantified. The expression level of the different PKCs was analyzed by Western blotting with an anti-Xpress antibody, and anti-actin immunoblotting was used as a control for protein loading (lower panels).

FIG. 3 is a schematic, and summary, of data from studies demonstrating that PKCθ is functionally coupled to CD28 costimulation, as described in detail in Example 1, below. FIG. 3a: Jurkat T cells were transfected with an empty vector (−) or wild type PKCθ (+) together with CD28RE/AP-1-Luc. After 20 hr, cells were stimulated for an additional 6 hr with CD3- and/or CD28-specific antibodies. Normalized luciferase activity in cell lysates was quantified as in FIG. 2. FIG. 3b: CD28RE/AP-1-Luc activity in Jurkat T cells cotransfected with an empty vector (−) or with a c-Myc-tagged Tat plasmid together with constitutively active PCK-θ, -α, -ζ or -ε mutants. The expression level of Tat or PKCθ was analyzed by Western blotting, and equal protein loading was confirmed by anti-actin immunoblotting (bottom panels).

FIG. 4 is a schematic, and summary, of data from studies demonstrating that inhibition of PKCθ blocks CD28 costimulation, as described in detail in Example 1, below. FIG. 4a: Jurkat T cells were cotransfected with CD28RE/AP-1-Luc (10 μg) and β-galactosidase plasmid (2 μg) reporters. Twenty hr later, cells were stimulated for 10 min with CD3- plus CD28-specific antibodies, in the presence or absence of rottlerin (Rott; 30 μM) or Gö6976 (Gö; 0.5 μM). Luciferase activity in cell lysates was determined. FIG. 4b Jurkat cells were incubated (15 min at 37° C.) in the absence or presence of rottlerin or Gö6976, and then stimulated with anti-CD3/CD28 antibodies or with TNFα (10 ng/ml) for the indicated times. Nuclear and cytoplasmic extracts were prepared, and protein from each fraction (5 μg) was analyzed by Western blotting with an anti-RelA antibody. FIG. 4c: Wild-type PKCθ-transfected Jurkat T cells were stimulated as in (a). Cell lysates were immunoprecipitated (IP) with normal rabbit serum (NRS) or with an anti-PKCθ antibody, and the in vitro enzymatic activity of PKCθ was measured in the presence or absence of rottlerin or Gö6976 (top panel). The membrane was immunoblotted with a PKCθ-specific antibody (bottom panel).

FIG. 5 is a schematic, and summary, of data from studies demonstrating that NF-κB activation induced by PKCθ is mediated by IKKβ/IκBα, as described in detail in Example 1, below. Jurkat cells were transfected with an empty vector (−) or constitutively active PKCθ (10 μg) together with NF-κB-Luc (FIG. 5a) CD28RE/AP-1-Luc (FIG. 5b), or AP-1-Luc (FIG. 5c) reporter constructs (5 μg each). The cells were cultured for 16 hr with the indicated concentrations of the kinase substrate MG132 (0, 5, 10, 20 μM), lysed, and luciferase activity was quantified. FIG. 5d: Jurkat cells were transfected with wild type IKKα (5 μg) or IKKβ (2 μg) together with an empty vector (0) or increasing amounts of constitutively active PKCθ. Twenty-four hr later, the cells were stimulated with anti-CD3/CD28 antibodies or with TNFα. Immunoprecipitated IKKα or IKKβ were subjected to an in vitro kinase assay. Phosphorylated GST-IκBα/1-62 was detected by autoradiography (top panels). The same membrane was immunoblotted with anti-c-Myc or anti-Flag antibodies (middle panels). Bottom panels show the expression level of PKCθ-A/E. FIG. 5e: Jurkat cells were transfected with an empty vector or with constitutively active PKCθ (10 μg) in the absence or presence of increasing amounts of kinase inactive IKKα or IKKβ mutants, together with a CD28RE/AP-1-Luc reporter (5 μg). After 24 hr, cells were lysed and normalized luciferase activity was determined. FIG. 5f: The expression level of the transfected IKKs or PKCθ was assessed by Western blotting using c-Myc-(IKKα), Flag-(IKKβ), or PKCθ-specific antibodies.

Like reference symbols in the various drawings indicate like elements.

### DETAILED DESCRIPTION

The invention provides methods for identifying agents that modulate various activities of protein kinase C theta (PKCθ) polypeptides. PKCθ is a necessary TCR/CD28 costimulatory signal and is essential for activation of the NF-κB cascade in T cells. The relatively selective expression (in lymphocytes, particularly T cells, and muscle cells) and essential function of PKCθ in T cell activation and survival demonstrate that the methods of the invention that are designed to selectively block the function of PKCθ in cells may be therapeutically useful in several scenarios. For example, since TCR engagement in the absence of CD28 costimulation can lead to T cell anergy (see, e.g., Chambers (1999) Curr Opin. Cell Biol. 11:203-210), inhibition of PKCθ can ablate the CD28 costimulatory signal and, therefore, promote T cell anergy. PKCθ inhibition can also abolish a T cell survival signal and, therefore, promote the apoptosis of activated self-reactive T cells, e.g., in autoimmune diseases. Lastly, since NF-κB activation appears to be necessary for the efficient replication of HIV-1 in T cells (see, e.g., Alcami (1995) EMBO J. 14:1552-1560, interfering with the function of PKCθ can inhibit viral replication in activated T cells. Accordingly, the methods of the invention (providing for the inhibition of PKCθ activity, and methods for identifying inhibitors of PKCθ activity) provide for ablation of the CD28 costimulatory signal in T cells, abolishing of a T cell survival signal, and promote the apoptosis of activated self-reactive T cells, e.g., in autoimmune diseases.

PKCθ plays an essential role in the activation of mature T cells by activating the NF-κB and AP-1 signaling cascades, which are required for IL-2 production and subsequent proliferation. The fact that these responses are defective in PKCθ-deficient mice indicates that this role is non-redundant and cannot be compensated by other PKC enzymes expressed in T cells. Studies demonstrated that the recruitment of PKCθ to the membrane, cytoskeleton and/or lipid rafts is regulated by a novel Vav/Rac pathway, which is largely independent of PLCγ1 activation. While the invention is not limited by any particular mechanism, these data suggest that selective recruitment of PKCθ to the immunological synapse is mediated by some scaffold protein that is either a cytoskeletal protein or a cytoskeleton-associated protein. Accordingly, in the methods of the invention, PKCθ activity can be determined by measuring its interactions or binding to a cytoskeletal protein or a cytoskeleton-associated protein.

The interaction between PKCθ and scaffold proteins represents a highly specific target for inducing selective immunosuppression of T cells in clinical situations. Accordingly, methods of the invention providing for blocking this interaction should complement strategies based on direct inhibition of the enzymatic activity of PKCθ, and offer several potential advantages over commonly used immunosuppressive drugs such as cyclosporin A and FK506. In addition, the methods of the invention provide for immunosuppression (e.g., to prevent transplant rejection or graft-vs.-host disease in bone marrow transplant recipients) by inhibition of PKCθ activity or PKCθ's essential translocation to the T cell synapse in antigen-stimulated T cells.

PKCθ provides a relatively selective survival signal that prevents T cell apoptosis via the process of activation-induced cell death (AICD). Thus, the methods of the invention, providing for inhibition of PKCθ function, can be used to promote the death of activated self-reactive T cells, which cause autoimmune diseases. Activation of NF-κB appears to be essential for productive HIV-1 replication in human T cells. Thus, the methods of the invention, providing for inhibition of PKCθ function, can severely reduce the replication of HIV-1 in T cells of infected individuals.

Functional interactions between Vav and PKCθ are required for TCR-induced T cell activation. The hematopoietic cell-specific signaling protein Vav, and the T cell-expressed PKCθ play an early and important role in the TCR/CD28-induced stimulation of MAP kinases and activation of the IL-2 gene. In addition, Vav is also essential for actin cytoskeleton reorganization and TCR capping. Finding that there is a novel functional interaction between Vav and PKCθ during early T cell activation, the invention provides methods for inhibiting the interaction between Vav and PKCθ, e.g., by providing methods to identify inhibitors of this interaction.

Studies found that intact PKCθ function was selectively required in a Vav signaling pathway that mediates the TCR/CD28-induced activation of JNK and the IL-2 gene and the upregulation of CD69 expression. It was also found that the requirement for functional PKCθ dissociated two distinct Vav signaling pathways, i.e., a PKCθ-dependent growth regulatory pathway and a PKCθ-independent signaling cascade which regulates the actin cytoskeleton. It was also found that although Vav did not interact physically with PKCθ, it promoted its translocation from the cytosol to the membrane and cytoskeleton, and induced its enzymatic activation in a CD3/CD28-initiated pathway that was dependent on Rac and on actin cytoskeleton reorganization. These findings revealed a novel and essential role for the Vav/Rac pathway, i.e., promoting the recruitment of PKCθ to the T cell synapse and its activation, processes that are essential for T cell activation and IL-2 production. Thus, the invention provides methods for identifying inhibitors of PKCθ activity by determining the ability of a test agent to inhibit Vav/PKCθ interaction.

PKCθ mediates a selective T cell survival signal via phosphorylation of BAD, a Bcl-2 family member. PKC-activating phorbol esters protect various cell types, including T cells, from apoptosis induced by the interaction of Fas with its ligand. However, before the instant invention, the mechanism of this protective effect as well as the identity of the PKC isoform(s) involved in this process were poorly understood. Studies analyzed the role of PKCθ selectively expressed in T lymphocytes in protecting T cells from Fas-mediated apoptosis. It was found that Fas triggering led to a selective and transient activation of PKCθ, which was later followed by caspase-dependent cleavage of the enzyme and proteasome-mediated degradation and inactivation of its catalytic fragment. These events preceded the onset of cell apoptosis. A PKCθ-selective inhibitor (rottlerin), but not an inhibitor of Ca²⁺-dependent PKC enzymes (Gö6976), synergized with an anti-Fas antibody to induce rapid T cell apoptosis and, conversely, transient overexpression of an active form of PKCθ selectively protected T cells from Fas-mediated apoptosis. NF-κB activation did not appear to play a major role in the PKCθ-mediated protective effect. Thus, the invention provides methods for identifying inhibitors of PKCθ activity by determining the ability of a test agent to inhibit PKCθ selective protection of T cells from Fas-mediated apoptosis.

It was also found that the distant Bcl-2 family member, BAD, mediates at least in part the anti-apoptotic effect of PKCθ. Triggering modes known to induce PKCθ activation, i.e., Fas or combined CD3/CD28 ligation, induced phosphorylation of BAD on Ser-136 (and to a lesser extent on Ser-112), which was selectively blocked by rottlerin. Furthermore, PKCθ selectively induced phosphorylation of BAD on the same serine residues both in vitro and in intact T cells. These findings indicated that PKCθ plays a selective role in protecting T cells from apoptosis via a pathway that involves BAD and, moreover, that the balance between PKCθ-dependent survival signals induced by CD3/CD28 costimulation and Fas-induced death signal may regulate the survival and death of T cells. Thus, the invention provides methods for identifying inhibitors of PKCθ activity by determining the ability of a test agent to inhibit BAD phosphorylation by PKCθ or a BAD activity.

TCR/CD28 costimulation induces selective PKCθ translocation to glycolipid-enriched membrane lipid microdomains. PKCθ is a novel Ca²⁺-independent PKC isoform, which is selectively expressed in T lymphocytes and skeletal muscle. In T cells, PKCθ plays an essential role in transcriptional activation of the IL-2 gene via selective stimulation of two transcription factors, AP-1 and NF-κB. However, before the instant invention, it was not known how PKCθ is regulated and becomes coupled to the TCR signaling machinery. Studies found that upon T cell activation by anti-CD3/CD28 antibodies, PKCθ translocated to detergent-insoluble glycolipid-enriched membrane domains known as “membrane rafts.” The translocation of PKCθ to membrane rafts was dependent on its regulatory domain, but did not require the catalytic domain. In intact T cells, PKCθ translocated to membrane patches induced by antibody-mediated cross-linking of membrane-bound cholera toxin. Similarly, engagement of antigen-specific, TCR-transgenic T cells with peptide-bound antigen-presenting cells (APCs) led to colocalization of PKCθ and membrane rafts at the contact area, i.e., the immunological synapse. The translocation of PKCθ to lipid rafts required Lck, but not ZAP-70, and the raft-resident fraction of PKCθ was physically associated with Lck. Consistent with the failure of the PKCθ catalytic domain (PKCθ-C) to localize to lipid rafts, it also failed to activate NF-κB. However, addition of an Lck-derived membrane and lipid raft localization sequence to PKCθ-C caused it to translocate to the rafts and activate NF-κB in anti-CD3/CD28-stimulated T cells. An agent that disrupts lipid rafts abolished the CD3/CD28-induced, Lck-mediated tyrosine phosphorylation of PKC, and also inhibited the activation of NF-κB. These results indicated that T cell activation induces PKCθ translocation to membrane rafts, which localize to the T cell synapse in Ag-stimulated T cells. Moreover, this translocation appears to be important for the physiological function of PKCθ. Thus, the invention provides methods for identifying inhibitors of PKCθ activity by determining the ability of a test agent to inhibit PKCθ translocation to membrane rafts.

PLCγ1-independent membrane translocation and activation of PKCθ in T cells: PKCθ is a Ca²⁺-independent member of the PKC family, which plays an essential role in mature T cell activation and proliferation via activation of the transcription factors AP-1 and NF-κB, both of which are required for induction of the IL-2 gene. Antigen stimulation leads to selective recruitment of PKCθ, but not other T cell-expressed PKCs, to the core region of the supramolecular activation complex (SMAC) or the immunological synapse. As discussed above, PKCθ membrane recruitment and activation are regulated by a novel Vav/Rac pathway. Further analysis of this mechanism investigated the contribution of the conventional, PLCγ1-mediated PKC activation pathway to PKCθ membrane translocation and catalytic activation.

Using three independent approaches (including a genetic approach) and a combination of biochemical and confocal microscopy analysis, it was found that, in contrast to PKCα, the membrane recruitment and activation of PKCθ are, to a large extent, PLCγ1-independent in both Jurkat T cells or activated human peripheral blood T cells. Conversely, a pharmacological inhibitor of phosphatidylinositol 3-kinase blocked the membrane translocation of PKCθ, but not of PKCα. In addition, PKCθ co-clustered with polymerized actin in activated T cell membranes, and this clustering was markedly reduced in Vav-deficient primary T cells. These results further support the existence of a non-conventional, PLCγ1-independent pathway, which mediates the selective recruitment of PLCγ1-independent to the immunological synapse and its activation. This mechanism could potentially represent a highly specific drug target for immunosuppression in T cells. Thus, the invention provides methods for identifying inhibitors of PKCθ activity by determining the ability of a test agent to inhibit PKCθ co-clustering with polymerized actin in activated T cell membranes.

Regulation of PKCθ function during T cell activation by Lck-mediated tyrosine phosphorylation: PKCθ in T cells co-localizes with the TCR/CD3 complex in antigen-stimulated T cells and is involved in the transcriptional activation of the IL-2 gene. It was found that PKCθ is tyrosine-phosphorylated in Jurkat T cells upon TCR/CD3 activation. The Src-family protein tyrosine kinase, Lck, was critical in TCR-induced tyrosine phosphorylation of PKCθ. Lck phosphorylated, and was associated with, the regulatory domain of PKCθ both in vitro and in intact cells. This association was constitutive, but it was enhanced by T cell activation, with both SH2 and SH3 domains of Lck contributing to it. Tyrosine 90 (Tyr-90) in the regulatory domain of PKCθ was identified as the major phosphorylation site by Lck. A constitutively active mutant of PKCθ (A148E) could enhance proliferation of Jurkat T cells and synergized with ionomycin to induce nuclear factor of T cells (NFAT) activity. However, mutation of Tyr-90 into phenylalanine markedly reduced (or abolished) these activities. These results suggest that Lck plays an important role in tyrosine phosphorylation of PKCθ, which may in turn modulate the physiological functions of PKCθ during TCR-induced T cell activation. Thus, the invention provides methods for identifying inhibitors of PKCθ activity by determining the ability of a test agent to inhibit PKCθ tyrosine-phosphorylation in Jurkat T cells upon TCR/CD3 activation. The invention also provides methods for identifying inhibitors of PKCθ activity by determining the ability of a test agent to inhibit Lck phosphorylation and association with the regulatory domain of PKCθ both in vitro and in intact cells.

The functional relationship between PKCθ and CD28 co-stimulation was determined, as described in Example 1, below. This relationship plays an essential role in TCR-mediated (interleukin-2) IL-2 production. The studies described in Example 1 demonstrate that PKCθ is functionally coupled to CD28 co-stimulation by virtue of its selective ability to activate the CD28RE/AP-1 element in the IL-2 gene promoter.

First described are studies demonstrating that CD28 co-stimulation enhanced the membrane translocation and catalytic activation of PKCθ. The next studies described demonstrate that among several PKC isoforms, PKCθ was the only one capable of activating NF-κB or CD28RE/AP-1 reporters in T cells (but not in 293T cells). Next described are studies demonstrating that wild type PKCθ synergized with CD28/CD3 signals to activate CD28RE/AP-1. In addition, PKCθ selectively synergized with Tat to activate a CD28RE/AP-1 reporter. Next described are studies demonstrating that CD3/CD28-induced CD28RE/AP-1 activation and NF-κB nuclear translocation were blocked by a selective PKCθ inhibitor. Lastly described are studies demonstrating that PKCθ-mediated activation of the same reporter was inhibited by the proteasome inhibitor MG132 (which blocks IκB degradation), and was found to involve IκB-kinase β (IKKβ). These findings identify a novel PKCθ-mediated pathway for the costimulatory action of CD28, which involves activation of the IKKβ/IκB/NF-κB signaling cascade.

Definitions

Unless defined otherwise, all technical and scientific terms used herein have the meaning commonly understood by a person skilled in the art to which this invention belongs. As used herein, the following terms have the meanings ascribed to them unless specified otherwise.

The term “antibody” or “Ab” includes both intact antibodies having at least two heavy (H) chains and two light (L) chains inter-connected by disulfide bonds and antigen binding fragments thereof, or equivalents thereof, either isolated from natural sources, recombinantly generated or partially or entirely synthetic. Examples of antigen binding fragments include, e.g., Fab fragments, F(ab′)2 fragments, Fd fragments, dAb fragments, isolated complementarity determining regions (CDR), single chain antibodies, chimeric antibodies, humanized antibodies, human antibodies made in non-human animals (e.g., transgenic mice) or any form of antigen binding fragment.

The terms “array” or “microarray” or “DNA array” or “nucleic acid array” or “biochip” as used herein is a plurality of target elements, each target element comprising a defined amount of one or more nucleic acid molecules, including the nucleic acids of the invention, immobilized a solid surface for hybridization to sample nucleic acids, as described in detail, below. The polypeptides (e.g., a PKCθ polypeptide) or nucleic acids used in the screening methods of the invention can be incorporated into any form of microarray, as described, e.g., in U.S. Pat. Nos. 6,045,996; 6,022,963; 6,013,440; 5,959,098; 5,856,174; 5,770,456; 5,556,752; 5,143,854.

The term “pharmaceutical composition” refers to a composition suitable for pharmaceutical use in a subject (including human or veterinary). The pharmaceutical compositions of this invention are formulations that comprise a pharmacologically effective amount of a composition comprising, e.g., a dominant negative or dominant positive PKCθ polypeptide, a nucleic acid comprising a PKCθ antisense sequence capable of modulating the expression of a PKCθ polypeptide in a cell, a PKCθ-specific antibody, a rottlerin or a functional equivalent thereof, a composition that inhibits binding of a lipid cofactor to PKCθ, or a composition that binds a nucleic acid regulating PKCθ expression.

The term “expression cassette” refers to any recombinant expression system for the purpose of expressing a nucleic acid sequence of the invention in vitro or in vivo, constitutively or inducibly, in any cell, including, in addition to mammalian (particularly human) cells, insect cells, plant cells, prokaryotic cells, yeast, or fungal cells. The term includes linear or circular expression systems. The term includes all vectors. The cassettes can remain episomal or integrate into the host cell genome. The expression cassettes can have the ability to self-replicate or not, i.e., drive only transient expression in a cell. The term includes recombinant expression cassettes that contain only the minimum elements needed for transcription of a recombinant nucleic acid, e.g., SEQ ID NO:1.

The term “heterologous” when used with reference to a nucleic acid, indicates that the nucleic acid is in a cell or plant where it is not normally found in nature; or, comprises two or more subsequences which are not found in the same relationship to each other as normally found in nature, or is recombinantly engineered so that its level of expression, or physical relationship to other nucleic acids or other molecules in a cell, or structure, is not normally found in nature. For instance, a heterologous nucleic acid is typically recombinantly produced, having two or more sequences from unrelated genes arranged in a manner not found in nature; e.g., a promoter sequence operably linked to a nucleic acid. As another example, the invention provides recombinant constructs (expression cassettes, vectors, viruses, and the like) comprising various combinations of promoters and sequence expressing PKCθ polypeptides and reporter constructs.

The term “nucleic acid” or “nucleic acid sequence” refers to a deoxy-ribonucleotide or ribonucleotide oligonucleotide, including single- or double-stranded forms, and coding or non-coding (e.g., “antisense”) forms. The term encompasses nucleic acids containing known analogues of natural nucleotides. The term also encompasses nucleic-acid-like structures with synthetic backbones. The term nucleic acid is used interchangeably with gene, DNA, RNA, cDNA, mRNA, oligonucleotide primer, probe and amplification product.

As used herein the terms “polypeptide,” “protein,” and “peptide” are used interchangeably and include compositions of the invention that also include “analogs,” or “conservative variants” and “mimetics” (e.g., “peptidomimetics”) with structures and activity that substantially correspond to the specified polypeptide, e.g., the PKCθ polypeptides used in the methods of the invention, including the exemplary sequence as set forth in SEQ ID NO:2. Thus, the terms “conservative variant” or “analog” or “mimetic” also refer to a polypeptide or peptide which has a modified amino acid sequence, such that the change(s) do not substantially alter the polypeptide's (the conservative variant's) structure and/or activity (e.g., PKCθ polypeptide activity), as defined herein. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity. Conservative substitution tables providing functionally similar amino acids are well known in the art. The terms “mimetic” and “peptidomimetic” refer to a synthetic chemical compound that has substantially the same structural and/or functional characteristics of the polypeptides of the invention (e.g., PKCθ polypeptide activity). The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetics' structure and/or activity.

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide.

As used herein, the term “promoter” includes all sequences capable of driving transcription of a coding sequence in a cell, including an insect cell, a plant cell, a mammalian cell, and the like. Thus, promoters used in the constructs of the invention include cis-acting transcriptional control elements and regulatory sequences that are involved in regulating or modulating the timing and/or rate of transcription of a coding sequence, e.g., for protein kinase C theta (PKCθ), or a detectable protein or an enzyme, e.g., luciferase.

Polypeptides and Peptides

The invention provides methods that use protein kinase C theta (PKCθ), such as the exemplary SEQ ID NO:2, and other polypeptides. Various peptides and peptidomimetics can be used as test compounds to be screened in the methods of the invention. Polypeptides used in the methods of the invention can be isolated from natural sources, be synthetic, or be recombinantly generated polypeptides. Peptides and proteins can be recombinantly expressed in vitro or in vivo. They can be made and isolated using any method known in the art.

Polypeptides used in the methods of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. The skilled artisan will recognize that individual synthetic residues and polypeptides incorporating mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Polypeptides and peptides incorporating mimetics can also be made using solid phase synthetic procedures, as described, e.g., by Di Marchi, et al., U.S. Pat. No. 5,422,426. Peptides and peptide mimetics used in the methods of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234. Modified peptides can be further produced by chemical modification methods, see, e.g., Belousov (1997) Nucleic Acids Res. 25:3440-3444; Frenkel (1995) Free Radic. Biol. Med. 19:373-380; Blommers (1994) Biochemistry 33:7886-7896.

Protein Kinase C Theta (PKCθ)

The invention provides methods that use a protein kinase C theta (PKCθ) polypeptide, including use of in vitro and in vivo recombinantly expressed PKCθ. As discussed above, any functional variation of a PKC θ can be used in the methods of the invention, including peptidomimetics and the like. See also U.S. Pat. No. 6,040,152.

In one aspect of the methods of the invention, human PKCθ is used. Isoforms and functional variations of PKCθ polypeptides, e.g., human PKCθ, can be used. For example, a human PKCθ as set forth in GenBank accession nos. NM 006257; XP 005709, can be used, such as the amino acid sequence (SEQ ID NO:2)

| | |
|---|---|
| MSPFLRIGLSNFDCGSCQSCQGEAVNPYCAVLVKEYVESENGQM | (SEQ ID NO:2). |
| YIQKKPTMYPPWDSTFDAHINKGRVMQIIVKGKNVDLISETTVELYSLAERCRKNNGK | |
| TEIWLELKPQGRMLMNARYFLEMSDTKDMNEFETEGFFALHQRRGAIKQAKVHHVKCH | |
| EFTATFFPQPTFCSVCHEFVWGLNKQGYQCRQCNAAIHKKCIDKVIAKCTGSAINSRE | |
| TMFHKERFKIDMPHRFKVYNYKSPTFCEHCGTLLWGLARQGLKCDACGMNVHHRCQTK | |
| VANLCGINQKLMAEALAMIESTQQARCLRDTEQIFREGPVEIGLPCSIKNEARPPCLP | |
| TPGKREPQGISWESPLDEVDKMCHLPEPELNKERPSLQIKLKIEDFILHKMLGKGSFG | |
| KVFLAEFKKTNQFFAIKALKKDVVLMDDDVECTMVEKRVLSLAWEHPFLTHMFCTFQT | |
| KENLFFVMEYLNGGDLMYHIQSCHKFDLSRATFYAAEIILGLQFLHSKGIVYRDLKLD | |
| NILLDKDGHIKIADFGMCKENMLGDAKTNTFCGTPDYIAPEILLGQKYNHSVDWWSFG | |
| VLLYEMLIGQSPFHGQDEEELFHSIRMDNPFYPRWLEKEAKDLLVKLFVREPEKRLGV | |
| RGDIRQHPLFREINWEELERKEIDPPFRPKVKSPFDCSNFDKEFLNEKPRLSFADRAL | |
| INSMDQNMFRNFSFMNPGMERLIS | |

In alternative aspects, a recombinant nucleic acid, e.g., an expression vector, encoding a polypeptide comprising a sequence as set forth in SEQ ID NO:2 is used. See also, e.g., Baier (1993) J. Biol. Chem. 268:4997-5004; Chang (1993) J. Biol. Chem. 268:14208-14214; Erdel (1995) Genomics 25:595-597.

Alternatively, a polypeptide encoded by a nucleic acid comprising a sequence as set forth in SEQ ID NO:1 can be used (see, e.g., GenBank accession nos. NM 006257; XM 005709)

| | | |
|---|---|---|
| 1 | tgctcgctcc agggcgcaac catgtcgcca tttcttcgga ttggcttgtc caactttgac | (SEQ ID NO:1); |
| 61 | tgcgggtcct gccagtcttg tcagggcgag gctgttaacc cttactgtgc tgtgctcgtc | |
| 121 | aaagagtatg tcgaatcaga gaacgggcag atgtatatcc agaaaaagcc taccatgtac | |
| 181 | ccaccctggg acagcacttt tgatgcccat atcaacaagg gaagagtcat gcagatcatt | |
| 241 | gtgaaaggca aaaacgtgga cctcatctct gaaaccaccg tggagctcta ctcgctggct | |
| 301 | gagaggtgca ggaagaacaa cgggaagaca gaaatatggt tagagctgaa acctcaaggc | |
| 361 | cgaatgctaa tgaatgcaag atactttctg gaaatgagtg acacaaagga catgaatgaa | |
| 421 | tttgagacgg aaggcttctt tgctttgcat cagcgccggg gtgccatcaa gcaggcaaag | |
| 481 | gtccaccacg tcaagtgcca cgagttcact gccaccttct tcccacagcc cacattttgc | |
| 541 | tctgtctgcc acgagtttgt ctggggcctg aacaaacagg gctaccagtg ccgacaatgc | |
| 601 | aatgcagcaa ttcacaagaa gtgtattgat aaagttatag caaagtgcac aggatcagct | |
| 661 | atcaatagcc gagaaaccat gttccacaag gagagattca aaattgacat gccacacaga | |
| 721 | tttaaagtct acaattacaa gagcccgacc ttctgtgaac actgtgggac cctgctgtgg | |
| 781 | ggactggcac ggcaaggact caagtgtgat gcatgtggca tgaatgtgca tcatagatgc | |
| 841 | cagacaaagg tggccaacct ttgtggcata aaccagaagc taatggctga agcgctggcc | |
| 901 | atgattgaga gcactcaaca ggctcgctgc ttaagagata ctgaacagat cttcagagaa | |
| 961 | ggtccggttg aaattggtct cccatgctcc atcaaaaatg aagcaaggcc gccatgttta | |
| 1021 | ccgacaccgg gaaaaagaga gcctcagggc atttcctggg agtctccgtt ggatgaggtg | |
| 1081 | gataaaatgt gccatcttcc agaacctgaa ctgaacaaag aaagaccatc tctgcagatt | |
| 1141 | aaactaaaaa ttgaggattt tatcttgcac aaaatgttgg ggaaaggaag ttttggcaag | |
| 1201 | gtcttcctgg cagaattcaa gaaaaccaat caatttttcg caataaaggc cttaaagaaa | |
| 1261 | gatgtggtct tgatggacga tgatgttgag tgcacgatgg tagagaagag agttctttcc | |
| 1321 | ttggcctggg agcatccgtt tctgacgcac atgttttgta cattccagac caaggaaaac | |
| 1381 | ctcttttttg tgatggagta cctcaacgga ggggacttaa tgtaccacat ccaaagctgc | |
| 1441 | cacaagttcg acctttccag agcgacgttt tatgctgctg aaatcattct tggtctgcag | |
| 1501 | ttccttcatt ccaaaggaat agtctacagg gacctgaagc tagataacat cctgttagac | |
| 1561 | aaagatggac atatcaagat cgcggatttt ggaatgtgca aggagaacat gttaggagat | |
| 1621 | gccaagacga ataccttctg tgggacacct gactacatcg ccccagagat cttgctgggt | |
| 1681 | cagaaataca accactctgt ggactggtgg tccttcgggg ttctccttta tgaaatgctg | |
| 1741 | attggtcagt cgcctttcca cgggcaggat gaggaggagc tcttccactc catccgcatg | |
| 1801 | gacaatccct tttacccacg gtggctggag aaggaagcaa aggaccttct ggtgaagctc | |
| 1861 | ttcgtgcgag aacctgagaa gaggctgggc gtgaggggag acatccgcca gcaccctttg | |
| 1921 | tttcgggaga tcaactggga ggaacttgaa cggaaggaga ttgacccacc gttccggccg | |
| 1981 | aaagtgaaat caccatttga ctgcagcaat ttcgacaaag aattcttaaa cgagaagccc | |
| 2041 | cggctgtcat ttgccgacag agcactgatc aacagcatgg accagaatat gttcaggaac | |
| 2101 | ttttccttca tgaaccccgg gatggagcgg ctgatatcct gaatcttgcc cctccagaga | |
| 2161 | caggaaagaa tttgccttct ccctgggaac tggttcaaga gacactgctt gggttccttt | |
| 2221 | ttcaacttgg aaaaagaaag aaacactcaa caataaagac tgagacccgt tcgcccccat | |
| 2281 | gtgactttat ctgtagcaga aaccaagtct acttcactaa tgacgatgcc gtgtgtctcg | |
| 2341 | tctcctgaca tgtctcacag acgctcctga agttaggtca ttactaacca tagttattta | |
| 2401 | cttgaaagat gggtctccgc acttggaaag gtttcaagac ttgatactgc aataaattat | |
| 2461 | ggctcttcac ctgggcgcca actgctgatc aacgaaatgc ttgttgaatc aggggcaaac | |
| 2521 | ggagtacaga cgtctcaaga ctgaaacggc cccattgcct ggtctagtag cggatctcac | |
| 2581 | tcagccgcag acaagtaatc actaacccgt tttattctat cctatctgtg gatgtataaa | |
| 2641 | tgctgggggc cagccctgga taggttttta tgggaattct ttacaataaa catagcttgt | |
| 2701 | acttg | |

or, SEQ ID NO:3,

| | | |
|---|---|---|
| 1 | ttccagggcg caaccatgtc gccatttctt cggattggct tgtccaactt tgactgcggg | (SEQ ID NO:3). |
| 61 | tcctgccagt cttgtcaggg cgaggctgtt aacccttact gtgctgtgct cgtcaaagag | |
| 121 | tatgtcgaat cagagaacgg gcagatgtat atccagaaaa agcctaccat gtacccaccc | |
| 181 | tgggacagca cttttgatgc ccatatcaac aagggaagag tcatgcagat cattgtgaaa | |
| 241 | ggcaaaaacg tggacctcat ctctgaaacc accgtggagc tctactcgct ggctgagagg | |
| 301 | tgcaggaaga acaacgggaa gacagaaata tggttagagc tgaaacctca aggccgaatg | |
| 361 | ctaatgaatg caagatactt tctggaaatg agtgacacaa aggacatgaa tgaatttgag | |
| 421 | acggaaggct tctttgcttt gcatcagcgc cggggtgcca tcaagcaggc aaaggtccac | |
| 481 | cacgtcaagt gccacgagtt cactgccacc ttcttcccac agcccacatt ttgctctgtc | |
| 541 | tgccacgagt ttgtctgggg cctgaacaaa cagggctacc agtgccgaca atgcaatgca | |
| 601 | gcaattcaca agaagtgtat tgataaagtt atagcaaagt gcacaggatc agctatcaat | |
| 661 | agccgagaaa ccatgttcca caaggagaga ttcaaaattg acatgccaca cagatttaaa | |
| 721 | gtctacaatt acaagagccc gaccttctgt gaacactgtg ggaccctgct gtggggactg | |
| 781 | gcacggcaag gactcaagtg tgatgcatgt ggcatgaatg tgcatcatag atgccagaca | |
| 841 | aaggtggcca acctttgtgg cataaaccag aagctaatgg ctgaagcgct ggccatgatt | |
| 901 | gagagcactc aacaggctcg ctgcttaaga gatactgaac agatcttcag agaaggtccg | |
| 961 | gttgaaattg gtctcccatg ctccatcaaa aatgaagcaa ggccgccatg tttaccgaca | |
| 1021 | ccgggaaaaa gagagcctca gggcatttcc tgggagtctc cgttggatga ggtggataaa | |
| 1081 | atgtgccatc ttccagaacc tgaactgaac aaagaaagac catctctgca gattaaacta | |
| 1141 | aaaattgagg attttatctt gcacaaaatg ttggggaaag gaagttttgg caaggtcttc | |
| 1201 | ctggcagaat tcaagaaaac caatcaattt ttcgcaataa aggccttaaa gaaagatgtg | |
| 1261 | gtcttgatgg acgatgatgt tgagtgcacg atggtagaga agagagttct ttccttggcc | |
| 1321 | tgggagcatc cgtttctgac gcacatgttt tgtacattcc agaccaagga aaacctcttt | |
| 1381 | tttgtgatgg agtacctcaa cggaggggac ttaatgtacc acatccaaag ctgccacaag | |
| 1441 | ttcgaccttt ccagagcgac gttttatgct gctgaaatca ttcttggtct gcagttcctt | |
| 1501 | cattccaaag gaatagtcta cagggacctg aagctagata acatcctgtt agacaaagat | |
| 1561 | ggacatatca agatcgcgga ttttggaatg tgcaaggaga acatgttagg agatgccaag | |
| 1621 | acgaatacct tctgtgggac acctgactac atcgccccag agatcttgct gggtcagaaa | |
| 1681 | tacaaccact ctgtggactg gtggtccttc ggggttctcc tttatgaaat gctgattggt | |
| 1741 | cagtcgcctt tccacgggca ggatgaggag gagctcttcc actccatccg catggacaat | |
| 1801 | cccttttacc cacggtggct ggagaaggaa gcaaaggacc ttctggtgaa gctcttcgtg | |
| 1861 | cgagaacctg agaagaggct gggcgtgagg ggagacatcc gccagcaccc tttgtttcgg | |
| 1921 | gagatcaact gggaggaact tgaacggaag gagattgacc caccgttccg gccgaaagtg | |
| 1981 | aaatcaccat ttgactgcag caatttcgac aaagaattct taaacgagaa gccccggctg | |
| 2041 | tcatttgccg acagagcact gatcaacagc atggaccaga atatgttcag gaacttttcc | |
| 2101 | ttcatgaacc ccgggatgga gcggctgata tcctgaatct tgcccctcca gagacaggaa | |
| 2161 | agaatttgcc ttctccctgg gaactggttc aagagacact gcttgggttc ctttttcaac | |
| 2221 | ttggaaaaag aaagaaacac tcaacaataa agactgagac ccgttcgccc ccatgtgact | |
| 2281 | tttatctgta gcagaaacca agtctacttc actaatgacg atgccgtgtg tctcgtctcc | |
| 2341 | tgacatgtct cacagacgct cctgaagtta ggtcattact aaccatagtt atttacttga | |
| 2401 | aagatgggtc tccgcacttg gaaaggtttc aagacttgat actgcaataa attatggctc | |
| 2461 | ttcacctggg cgccaactgc tgatcaatga aatgcttgtt gaatcagggg caaacggagt | |
| 2521 | acagacgtct caagactgaa acggccccat tgcctggtct agtagcggat ctcactcagc | |
| 2581 | cgcagacaag taatcactaa cccgttttat tctattccta tctgtggatg tgtaaatggc | |
| 2641 | tggggggcca gccctggata ggtttttatg ggaattcttt acaataaaca tagcttgt | |

Alternatively, genomic sequences can be used, e.g., in construct to express PKCθ recombinantly, see, e.g., Kofler (1998) Mol. Gen. Genet. 259:398-403, that described the genomic structure of the human PRKCQ gene that encodes the human PKCθ polypeptide. See also, Erdel (1995) supra.

In other aspects of the invention, other mammalian PKCθs are used, as, e.g., mouse PKCθ (see, e.g., GenBank accession nos. NM 008859; NP 032885; and Osada (1992) Mol. Cell. Biol. 12:3930-3938; Mischak (1993) FEBS Lett. 326:51-55).

Nucleic Acids, Expression Vectors and Transformed Cells

The invention provides recombinant nucleic acids comprising sequences coding for PKCθs, e.g., SEQ ID NO:1, and reporter constructs, including expression cassettes (e.g., vectors), cells and transgenic animals comprising the nucleic acids used in the methods of the invention. As the genes and vectors of the invention can be made and expressed in vitro or in vivo, the invention provides for a variety of means of making and expressing these genes and vectors. The invention can be practiced in conjunction with any method or protocol known in the art, which are well described in the scientific and patent literature. The nucleic acid sequences of the invention and other nucleic acids used to practice this invention, whether RNA, cDNA, genomic DNA, vectors, viruses or hybrids thereof, may be isolated from a variety of sources, genetically engineered, amplified, and/or expressed recombinantly. Any recombinant expression system can be used, including, in addition to mammalian cells, insect and bacterial cells, yeast or plant cell expression systems.

Alternatively, these nucleic acids can be synthesized in vitro by well-known chemical synthesis techniques, as described in, e.g., Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., generating mutations in sequences, subcloning, labeling probes, sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993). The nucleic acids used in the methods of the invention can be “operably linked” to a transcriptional regulatory sequence. “Operably linked” refers to a functional relationship between two or more nucleic acid (e.g., DNA) segments. For example, a promoter is operably linked to a coding sequence, such as a nucleic acid of the invention, if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. For example, in one embodiment, a promoter is operably linked to a nucleic acid sequence, e.g., SEQ ID NO:1, or a reporter construct.

Expression vectors capable of expressing the nucleic acids and polypeptides of the invention in animal cells, including mammalian cells, are well known in the art. Vectors which may be employed include recombinantly modified enveloped or non-enveloped DNA and RNA viruses, e.g., from baculoviridiae, parvoviridiae, picomoviridiae, herpesveridiae, poxviridae, adenoviridiae, picornnaviridiae or alphaviridae. Mammalian expression vectors can be derived from adenoviral, adeno-associated viral or retroviral genomes. Retroviral vectors can include those based upon murine leukemia virus (see, e.g., U.S. Pat. No. 6,132,731), gibbon ape leukemia virus (see, e.g., U.S. Pat. No. 6,033,905), simian immuno-deficiency virus, human immuno-deficiency virus (see, e.g., U.S. Pat. No. 5,985,641), and combinations thereof. Describing adenovirus vectors, see, e.g., U.S. Pat. Nos. 6,140,087; 6,136,594; 6,133,028; 6,120,764. See, e.g., Okada (1996) Gene Ther. 3:957-964; Muzyczka(1994) J. Clin. Invst. 94:1351; U.S. Pat. Nos. 6,156,303; 6,143,548 5,952,221, describing AAV vectors. See also 6,004,799; 5,833,993.

Transgenic Non-human Animals

The invention also provides transgenic non-human animals, including mammals, for use in the methods of the invention. Transgenic non-human mammals include, e.g., goats, rats and mice, comprising nucleic acids used to practice the methods of the invention. These animals can be used, e.g., as in vivo models to screen for modulators of PKCθ activity, or enzyme activity in vivo. Transgenic non-human animals can be designed and generated using any method known in the art; see, e.g., U.S. Pat. Nos. 6,156,952; 6,118,044; 6,111,166; 6,107,541; 5,959,171; 5,922,854; 5,892,070; 5,880,327; 5,891,698; 5,639,940; 5,573,933.

Antibodies

The invention provides antibodies that specifically bind to PKCθ, e.g., the exemplary SEQ ID NO:2. These antibodies can be used, e.g., to inhibit the activity of PKCθ, isolate PKCθ, to identify PKCθ in a sample, and the like. To generate antibodies, polypeptides or peptides (antigenic fragments of SEQ ID NO:2) can be conjugated to another molecule or can be administered with an adjuvant. The coding sequence can be part of an expression cassette or vector capable of expressing the immunogen in vivo (see, e.g., Katsumi (1994) Hum. Gene Ther. 5:1335-9). Methods of producing polyclonal and monoclonal antibodies are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif.; Goding, MONOCLONAL ANTIBODIES: PRINCIPLES AND PRACTICE (2d ed.) Academic Press, New York, N.Y. (1986); Harlow (1988) ANTIBODIES, A LABORATORY MANUAL, Cold Spring Harbor Publications, New York.

Antibodies also can be generated in vitro, e.g., using recombinant antibody binding site expressing phage display libraries, in addition to the traditional in vivo methods using animals. See, e.g., Huse (1989) Science 246:1275; Ward (1989) Nature 341:544; Hoogenboom (1997) Trends Biotechnol. 15:62-70; Katz (1997) Annu. Rev. Biophys. Biomol. Struct. 26:27-45. Human antibodies can be generated in mice engineered to produce only human antibodies, as described by, e.g., U.S. Pat. No. 5,877,397; 5,874,299; 5,789,650; and 5,939,598. B-cells from these mice can be immortalized using standard techniques (e.g., by fusing with an immortalizing cell line such as a myeloma or by manipulating such B-cells by other techniques to perpetuate a cell line) to produce a monoclonal human antibody-producing cell. See, e.g., U.S. Pat. Nos. 5,916,771; 5,985,615.

Formulation and Administration Pharmaceuticals

The pharmaceutical compositions of this invention are formulations that comprise a pharmacologically effective amount of a composition comprising, e.g., a dominant negative or dominant positive PKCθ polypeptide, a nucleic acid comprising a PKCθ antisense sequence capable of modulating the expression of a PKCθ polypeptide in a cell, a PKCθ-specific antibody, a rottlerin or a functional equivalent thereof, a composition that inhibits binding of a lipid cofactor to PKCθ, or a composition that binds a nucleic acid regulating PKCθ expression. These pharmaceuticals can be administered by any means in any appropriate formulation. Routine means to determine drug regimens and formulations to practice the methods of the invention are well described in the patent and scientific literature. For example, details on techniques for formulation, dosages, administration and the like are described in, e.g., the latest edition of Remington's Pharmaceutical Sciences, Maack Publishing Co, Easton Pa.

The formulations of the invention can include pharmaceutically acceptable carriers that can contain a physiologically acceptable compound that acts, e.g., to stabilize the composition or to increase or decrease the absorption of the pharmaceutical composition. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of any co-administered agents, or excipients or other stabilizers and/or buffers. Detergents can also used to stabilize the composition or to increase or decrease the absorption of the pharmaceutical composition. Other physiologically acceptable compounds include wetting agents, emulsifying agents, dispersing agents or preservatives that are particularly useful for preventing the growth or action of microorganisms. Various preservatives are well known, e.g., ascorbic acid. One skilled in the art would appreciate that the choice of a pharmaceutically acceptable carrier, including a physiologically acceptable compound depends, e.g., on the route of administration and on the particular physio-chemical characteristics of any co-administered agent.

In one aspect, the composition for administration comprises a pharmaceutically acceptable carrier, e.g., an aqueous carrier. A variety of carriers can be used, e.g., buffered saline and the like. These solutions are sterile and generally free of undesirable matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration and imaging modality selected.

The pharmaceutical formulations of the invention can be administered in a variety of unit dosage forms, the general medical condition of each patient, the method of administration, and the like. Details on dosages are well described in the scientific and patent literature, see, e.g., the latest edition of Remington's Pharmaceutical Sciences. The exact amount and concentration of pharmaceutical of the invention and the amount of formulation in a given dose, or the “effective dose” can be routinely determined by, e.g., the clinician. The “dosing regimen,” will depend upon a variety of factors, e.g., the general state of the patient's health, age and the like. Using guidelines describing alternative dosaging regimens, e.g., from the use of other imaging contrast agents, the skilled artisan can determine by routine trials optimal effective concentrations of pharmaceutical compositions of the invention. The invention is not limited by any particular dosage range.

The pharmaceutical compositions of the invention can be delivered by any means known in the art systemically (e.g., intravenously), regionally, or locally (e.g., intra- or peri-tumoral or intracystic injection) by, e.g., intraarterial, intratumoral, intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa), intra-tumoral (e.g., transdermal application or local injection). For example, intra-arterial injections can be used to have a “regional effect,” e.g., to focus on a specific organ (e.g., brain, liver, spleen, lungs).

The pharmaceutical formulations of the invention can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Therapeutic compositions can also be administered in a lipid formulation, e.g., complexed with liposomes or in lipid/nucleic acid complexes or encapsulated in liposomes, as in immunoliposomes directed to specific cells. These lipid formulations can be administered topically, systemically, or delivered via aerosol. See, e.g., U.S. Pat. Nos. 6,149,937; 6,146,659; 6,143,716; 6,133,243; 6,110,490; 6,083,530; 6,063,400; 6,013,278; 5,958,378; 5,552,157.

High-throughput Screening for Inhibitors of PKCθ

The invention provides methods for screening for agents that can modulate (e.g., inhibit or increase) an activity of a PKCθ polypeptide. The screening can be done in vitro or in vivo. For in vitro screening, large numbers of compounds can be quickly and efficiently tested using “high throughput screening (HTS)” methods. High throughput screening methods involve providing a library containing a large number of potential therapeutic compounds (“candidate compounds”). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

Use of combinatorial chemical libraries as a source of test agents are one means to assist in the generation of new chemical compound leads, i.e., compounds that inhibit an activity of a PKCθ polypeptide. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. Preparation and screening of combinatorial chemical libraries are well known to those of skill in the art, see, e.g., U.S. Pat. Nos. 6,096,496; 6,075,166; 6,054,047; 6,004,617; 5,985,356; 5,980,839; 5,917,185; 5,767,238. Such combinatorial chemical libraries include, but are not limited to, peptide libraries (see, e.g., U.S. Pat. No. 5,010,175; Furka (1991) Int. J. Pept. Prot. Res., 37: 487-493, Houghton et al. (1991) Nature, 354: 84-88). Other chemistries for generating chemical diversity libraries include, but are not limited to: peptoids (see, e.g., WO 91/19735), encoded peptides (see, e.g., WO 93/20242), random bio-oligomers (see, e.g., WO 92/00091), benzodiazepines (see, e.g., U.S. Pat. No. 5,288,514), diversomers such as hydantoins, benzodiazepines and dipeptides (see, e.g., Hobbs (1993) Proc. Nat. Acad. Sci. USA 90: 6909-6913), vinylogous polypeptides (see, e.g., Hagihara (1992) J. Amer. Chem. Soc. 114: 6568), nonpeptidal peptidomimetics with a Beta-D-Glucose scaffolding (see, e.g., Hirschmann (1992) J. Amer. Chem. Soc. 114: 9217-9218), analogous organic syntheses of small compound libraries (see, e.g., Chen (1994) J. Amer. Chem. Soc. 116: 2661), oligocarbamates (see, e.g., Cho (1993) Science 261:1303), and/or peptidyl phosphonates (see, e.g., Campbell (1994) J. Org. Chem. 59: 658). See also Gordon (1994) J. Med. Chem. 37:1385; for nucleic acid libraries, peptide nucleic acid libraries, see, e.g., U.S. Pat. No. 5,539,083; for antibody libraries, see, e.g., Vaughn (1996) Nature Biotechnology 14:309-314; for carbohydrate libraries, see, e.g., Liang et al. (1996) Science 274: 1520-1522, U.S. Pat. No. 5,593,853; for small organic molecule libraries, see, e.g., for isoprenoids U.S. Pat. No. 5,569,588; for thiazolidinones and metathiazanones, U.S. Pat. No. 5,549,974; for pyrrolidines, U.S. Pat. Nos. 5,525,735 and 5,519,134; for morpholino compounds, U.S. Pat. No. 5,506,337; for benzodiazepines U.S. Pat. No. 5,288,514.

Devices for the preparation of combinatorial libraries are commercially available (see, e.g., U.S. Pat. No. 6,045,755; 5,792,431; 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). A number of robotic systems have also been developed for solution phase chemistries. These systems include automated workstations, e.g., like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Antisense Sequences

The pharmaceutical compositions of this invention can comprise a pharmacologically effective amount of a composition comprising a nucleic acid comprising a PKCθ antisense sequence capable of modulating the expression of a PKCθ polypeptide in a cell, including, e.g., SEQ ID NO:1 or antisense fragments thereof. Identification of effective antisense sequences, e.g., oligonucleotides that can inhibit the expression of a message or a gene in vivo, has become a matter of routine screening. Synthesis of effective synthetic antisense oligonucleotides, and means for their delivery in vivo, has also become routine. See, e.g., U.S. Pat. Nos. 6,165,990; 6,165,791; 6,165,790; 6,165,789; 5,814,500; 5,417,978; 5,627,274.

### EXAMPLES

The following example is offered to illustrate, but not to limit the claimed invention.

### Example 1

Functions of Protein Kinase C theta (PKCθ)

The following example describes studies demonstrating the role of protein kinase C theta (PKCθ) in various aspects of T cell activation, apoptosis, anergy and other aspects of cell physiology.

Plasmids: The 4xRE/AP-luciferase reporter described by Shapiro (1997) Mol. Cell. Biol. 17:4051-4058, was obtained from A. Weiss (University of California, San Francisco, Calif.). The NF-κB- and AP-1-luciferase reporter plasmids were obtained from M. Karin (University of California, San Diego, Calif.). The pEF4-LacZ reporter plasmid was obtained from Invitrogen (San Diego, Calif.). A Tat cDNA was generated by reverse transcription of RNA extracted from HIV-1/Lai-infected cells. The two exons of Tat (aa 1-86) were subcloned by RT-PCR into the EcoRI and XbaI sites of the pEF4/myc-His mammalian expression vector (Invitrogen). The stop codon was removed and the insert was subcloned in frame to the C-terminal c-Myc tag. IKKα and IKKβ were excised from the pEV3S and pcDNA3.1 vectors, respectively (obtained from W. Greene, Gladstone Institute, San Francisco, Calif.) by digestion with KpnI/NheI and XbaI/HindIII, respectively, blunted and subcloned into the EcoRV site of the pEF4/myc-His vector. The IKKβ plasmid encodes a C-terminal Flag epitope derived from the original vector. The cDNAs encoding human wild type or constitutively active mutants of human PKCθ and PKCα, rat PKCS, or mouse PKCζ have been described by Villalba (1999) J. Immunol. 163:5813-5819. Xpress™ epitope-tagged versions of these PKCs were generated using the pEF4/His mammalian expression vector (Invitrogen).

Cell Culture, Transfection, and Reporter Assays. Human leukemic Jurkat (E6. 1) and 293T cells were cultured. Cells in a logarithmic growth phase were transfected with the indicated amounts of plasmid DNAs by electroporation. Cells were stimulated for the indicated times with combinations of anti-CD3 and/or CD28 antibodies (Pharmingen) or with TNFα (Genzyme). In some experiments, the cells were treated with rottlerin, Gö6976 (both from Calbiochem), or MG132 (Biomol). Transfected Jurkat cells were harvested after 24 hr, washed twice with PBS, lysed, and luciferase or β-galactosidase activities were determined. The results were expressed as arbitrary luciferase units normalized to β-galactosidase activity in the same cells. All experiments were performed at least twice with similar results.

Immunoprecipitation and Western Blotting: These procedures were performed using standard protocols. Briefly, cells were lysed, and the supernatants obtained after centrifugation were incubated with optimal concentrations of primary antibodies, followed by addition of protein G-plus-Sepharose (Pharmacia). Washed immunoprecipitates were dissolved in Laemli buffer, resolved by SDS-polyacrylamide gel electrophoresis, and transferred to nitrocellulose membranes, which were blocked with 5% dry milk. The membranes were incubated with blocking buffer containing optimal concentrations of blotting antibodies, washed, and incubated with horseradish peroxidase (HRP)-conjugated secondary anti-rabbit or -mouse IgG antibodies (Amersham). After washing, the blots were developed using an enhanced chemiluminescence kit (Amersham). As control for protein loading, samples were also immunoblotted with an anti-actin monoclonal antibody (ICN).

Kinase Assays. Endogenous PKCθ was immunoprecipitated using a polyclonal antibody (Santa Cruz Biotechnology), and transfected IαB-kinase a (IKKα) or IβB-kinase β (IKKβ) were immunoprecipitated using monoclonal antibodies specific for the c-Myc (Santa Cruz) or Flag (Sigma) epitopes, respectively. Immunoprecipitates were resuspended in 20 μl of the respective kinase buffers containing 5 μCi [γ-³²P] ATP and 1 μg myelin basic protein (MBP) or GST-IκBα/1-62 as substrates for PKC or IKK, respectively. Where indicated, rottlerin or Gö6976 were added to the PKC kinase reactions. Reactions were incubated for 20-30 min at 30° C. with gentle shaking, subjected to SDS-PAGE, transferred to nitrocellulose, and developed by autoradiography. [γ-³²P] ATP incorporation was determined using a STORM 860™ PhosphorImager (Molecular Dynamics). Nitrocellulose membranes were re-probed with the corresponding kinase- or tag-specific antibodies to determine expression levels of the immunoprecipitated kinases.

Subcellular Fractionation. To determine PKCθ redistribution, cells were fractionated into cytosolic or membrane fractions as described by Meller (1996) Mol. Cell. Biol. 16:5782-5791; and, SDS-PAGE-resolved proteins were immunoblotted with an HRP-conjugated anti-PKCθ monoclonal antibody (Transduction Laboratories). For NF-κB translocation, nuclear and cytoplasmic extracts were prepared and stored at −80° C. as described by Dejardin (1999) Oncogene 18:2567-2577. Extracts were resolved by SDS-PAGE, transferred to nitrocellulose, and immunoblotted with a polyclonal anti-RelA (p65) antibody (Santa Cruz).

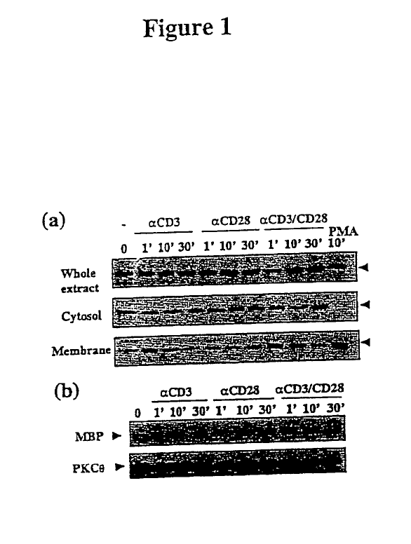

CD28 Co-stimulation Enhances the Translocation and Activity of PKCθ: T cell activation is associated with translocation of PKCθ to the membrane, and, more specifically, to the T cell synapse. In order to assess the contribution of CD28 co-stimulation to these events and to the activation of PKCθ, the effects of anti-CD3 and/or CD28 antibody stimulation on the localization and activity of PKCθ in T cells was compared. Costimulation of Jurkat T cells with both antibodies enhanced in parallel the translocation (FIG. 1a) and in situ catalytic activity (FIG. 1b) of PKCθ by comparison with either single stimulus. Both responses peaked at 1-10 min and declined after 30 min. These effects may be mediated by a Vav/Rac pathway that acts selectively on PKCθ, but not on other T cell-expressed PKC isoforms.

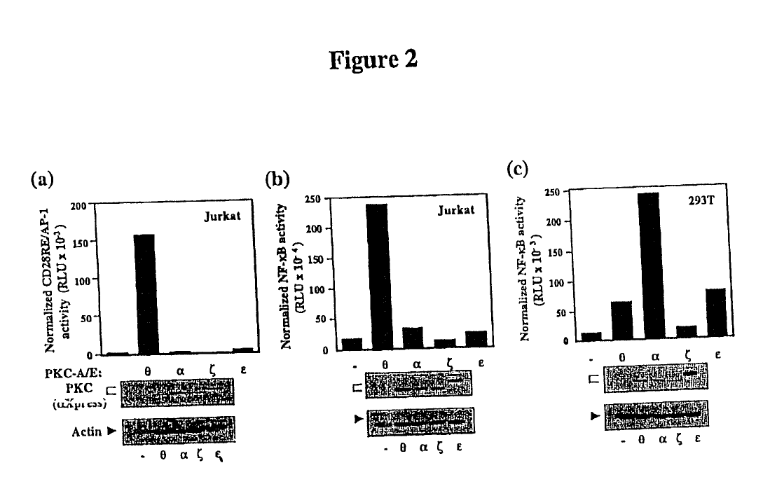

Selective Activation of NF-κB and CD28RE/AP-1 by PKCθ: CD28 is known to mediate its costimulatory function by activating the CD28RE/AP-1 element in the IL-2 gene promoter. Therefore, the role of PKCθ in activating this element was analyzed. As shown in FIG. 2a, a constitutively active mutant (A/E) of PKCθ, but not α, ζ or ε mutants, induced marked activation of the CD28RE/AP-1 reporter in transiently cotransfected T cells. As reported for anti-CD3/CD28 costimulation by Shapiro (1997) Mol. Cell. Biol. 17:4051-4058, and McGuire (1997) J. Immunol. 159:1319-1327, the effect of PKCθ required both NF-κB- and AP-1-binding sites since CD28RE/AP-1 reporter constructs in which either site was mutated were not activated by PKCθ.

Because the CD28RE/AP-1 element contains binding sites for both AP-1 and NF-κB, and Baier-Bitterlich (1996) Mol. Cell. Biol. 16:1842-1850, finding that PKCθ is a selective AP-1 activator in T cells, it was next determined whether PKCθ can also activate an isolated NF-κB reporter. The PKCθ-A/E mutant induced strong activity of the NF-κB-Luc reporter while other PKC isoforms induced very weak (α, ε) or no (ζ) activity (FIG. 2b). The PKCθ-induced NF-κB activity was not enhanced by additional stimulation with phorbol ester, suggesting that PKCθ is the predominant, if not exclusive, mediator of NF-κB activation. The effect of PKCθ on NF-κB was cell-specific, PKCθ (and PKCε) stimulated low NF-κB activity in 293T cells, while PKCα displayed the highest activity in these cells (FIG. 2c). All PKC isoforms tested were properly over-expressed in the cells (FIG. 2, bottom panels) and, furthermore, were functional as indicated by their ability to stimulate the activity of a cotransfected ERK2 reporter (Werlen (1998) EMBO J. 17:3101-3111).

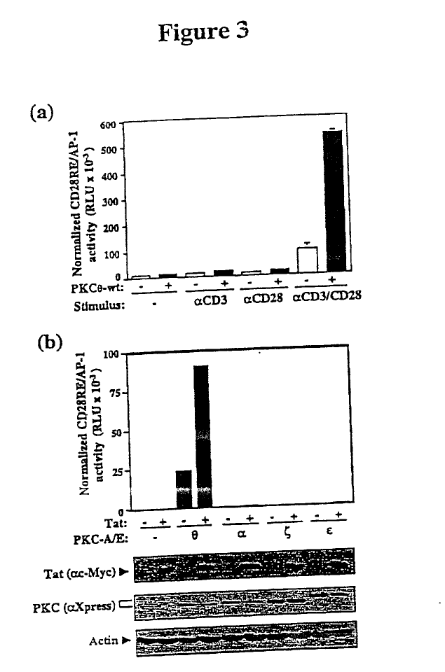

Several experiments were carried out to further establish the functional coupling of PKCθ to the CD3/CD28 co-stimulation pathway. First, wild-type PKCθ synergized with anti-CD3 plus -CD28 antibodies to activate a CD28RE/AP-1 element-containing reporter (FIG. 3a). Second, the ability of constitutively active PKC mutants to synergize with the HIV-1-derived protein, Tat, was tested. This was based on findings that HIV-1 infection or Tat overexpression synergizes with CD3/CD28 costimulation to superinduce the IL-2 and IL-8 genes; this effect is mediated by the action of Tat on the CD28RE element, as described by McGuire (1997) J. Immunol. 159:1319-1327; Ott (1997) Science 275:1481-1485. Among four PKC isoforms tested, only PKCθ could synergize with cotransfected Tat to activate the CD28RE/AP-1 promoter-driven reporter (FIG. 3b). These results suggest that PKCθ is functionally coupled to CD3/CD28 costimulation.

Inhibition of CD28RE/AP-1 Activation by a Selective PKCθ Inhibitor: The importance of PKCθ in CD28RE and NF-κB activation was assessed by analyzing the effect of rottlerin, originally found to be a selective PKCδ inhibitor by Gschwendt (1994) Biochem. Biophys. Res. Commun. 199:93-98 (see also, e.g., Parmer (1997) Cell Growth Differ. 8:327-334). Villalba (1999) J. Immunol. 163:5813-5819, recently found that rottlerin also inhibits PKCθ function in vitro and in intact T cells and, furthermore, that these cells do not express PKCδ.

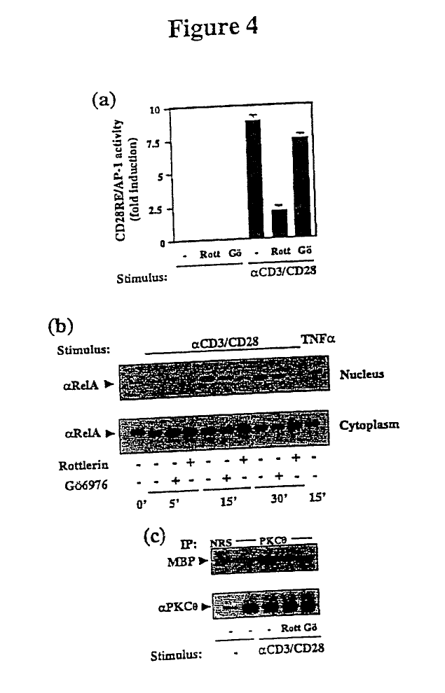

Rottlerin inhibited the anti-CD3/CD28-stimulated activity of CD28RE/AP-1 by approximately 80% (FIG. 4a), and essentially blocked the receptor-stimulated nuclear translocation of RelA (p65) (FIG. 4b), an NF-κB component that is known to bind to the CD28RE/AP-1 element (Ghosh (1993) Proc. Natl. Acad. Sci. USA 90:1696-1700). Rottlerin did not inhibit NF-κB activity induced by tumor necrosis factor a (TNFα), indicating that CD3/CD28 and TNFα signals activate NF-κB via distinct pathways. The specificity of these inhibitory effects is evident from the finding that Gö6976, a PKC inhibitor selective for Ca²⁺-dependent PKC isoforms (see, e.g., Martiny Baron (1993) J. Biol. Chem. 268:9194-9197), caused minimal inhibition of these functions. A control experiment confirmed that rottlerin was considerably more effective than Gö6976 in inhibiting CD3/CD28-induced PKCθ activity (FIG. 4c).

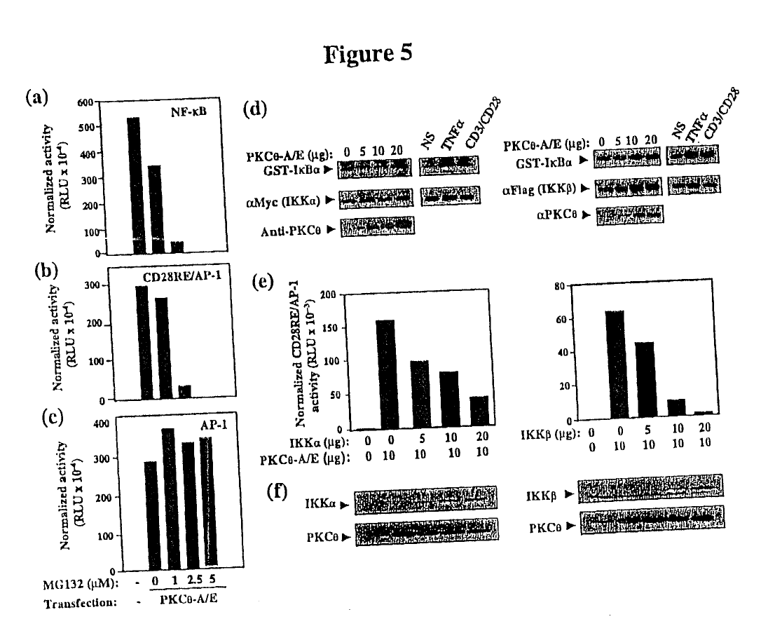

PKCθMediated NF-κB and CD28RE/AP-1 Activation Involves IκB and IKK. Additional experiments were conducted to elucidate the pathway leading from PKCθ to NF-κB activation, and further establish the physiological relevance of PKCθ in these events. FIG. 5 summarizes data from these studies, which demonstrated that NF-κB activation induced by PKCθ is mediated by IKKβ/IκBα. Jurkat cells were transfected with an empty vector (−) or constitutively active PKCθ (10 μg) together with NF-κB-Luc (FIG. 5a) CD28RE/AP-1-Luc (FIG. 5b), or AP-1-Luc (FIG. 5c) reporter constructs (5 μg each). The cells were cultured for 16 hr with the indicated concentrations of the protease inhibitor MG132 (0, 5, 10, 20 μM), lysed, and luciferase activity was quantified.

FIG. 5d: Jurkat cells were transfected with wild type IKKα (5 μg) or IKKβ (2 μg) together with an empty vector (0) or increasing amounts of constitutively active PKCθ. Twenty-four hr later, the cells were stimulated with anti-CD3/CD28 antibodies or with TNFα for 10 min. Immunoprecipitated IKKα or IKKβ were subjected to an in vitro kinase assay. Phosphorylated GST-IκBα/1-62 was detected by autoradiography (top panels). The same membrane was immunoblotted with anti-c-Myc or anti-Flag antibodies (middle panels). Bottom panels show the expression level of PKCθ-A/E.

FIG. 5e: Jurkat cells were transfected with an empty vector or with constitutively active PKCθ (10 μg) in the absence or presence of increasing amounts of kinase inactive IKKα or IKKβ mutants, together with a CD28RE/AP-1-Luc reporter (5 μg). After 24 hr, cells were lysed and normalized luciferase activity was determined.

FIG. 5f: The expression level of the transfected IKKs or PKCθ was assessed by Western blotting using c-Myc-(IKKα), Flag-(IKKβ), or PKCθ-specific antibodies.

The selective proteasome inhibitor, MG132, which prevents IκB degradation (see Palombella (1994) Cell 78:773-785), blocked in a dose-dependent manner the PKCθ-A/E-induced activation of NF-κB (FIG. 5a) and CD28RE/AP-1 (FIG. 5b), but not of AP-1 (FIG. 5c). Similar results were obtained with an IκB phosphorylation inhibitor, BAY 11-7082, indicating that IκB degradation is important. Next, the role of IKK in PKCθ-mediated CD28RE/AP-1 activation was determined. Similar to anti-CD3/CD28 stimulation, which has been reported to activate IKK by Harhaj (1998) J. Biol. Chem. 273:25185-25190, and Kempiak (1999) J. Immunol. 162:3176-3187, it was found that constitutively active PKCθ also induced significant activation of IβB-kinase β (IKKβ), but not IKKα, to an extent similar to that induced by TNFα or anti-CD3/CD28 stimulation (FIG. 5d).

The biological relevance of this activation is indicated by the finding that a dominant-negative IβB-kinase β (IKKβ) mutant, which can inhibit CD3/CD28-induced activation of a CD28RE/AP-1 promoter-driven reporter, as reported by Harhaj (1998) J. Biol. Chem. 273:25185-25190, Kempiak (1999) J. Immunol. 162:3176-3187, inhibited activation of the same reporter induced by PKCθ. A dominant negative IKKα mutant was less active (FIG. 5e), possibly reflecting its potential contribution to the formation of an IKKα/IKKβ heterodimer. The ability of CD3/CD28 stimulation, but not PKCθ, to activate IKKα suggests that CD3/CD28 signals may activate IKKα via a PKCθ-independent pathway, but the physiological significance of IKKα in the context of CD28RE/AP-1 induction remains unclear. Both IKKs and PKCθ were properly overexpressed in the cells (FIG. 5f).

Taken together, these results identify a novel PKCθ-mediated pathway for the costimulatory action of CD28. This pathway involves activation of the IKK/IβB-kinase β (IKKβ)/NF-κB signaling cascade, leading to stimulation of the combined CD28RE/AP-1 site in the IL-2 gene promoter. Accordingly, the invention provides novel means of screening for and identifying modulators of PKCθ polypeptides.

A number of embodiments of the invention have been described. Nevertheless, it will be understood that various modifications may be made without departing from the spirit and scope of the invention. Accordingly, other embodiments are within the scope of the following claims.

What is claimed is:

1. A method for identifying an agent that modulates an activity of a PKCθ polypeptide comprising:
(a) providing a PKCθ polypeptide and a test agent;
(b) contacting the PKCθ polypeptide with the test agent; and,
(c) determining the activity of the PKCθ polypeptide, wherein an increase or decrease in activity of the PKCθ polypeptide in the presence of the test agent thereby identifies the agent as a modulator of PKCθ polypeptide activity.

2. The method of claim 1, wherein the PKCθ comprises a polypeptide encoded by a nucleic acid comprising a sequence as set forth in SEQ ID NO:1 or comprising an amino acid sequence as set forth in SEQ ID NO:2.

3. The method of claim 1, wherein the PKCθ polypeptide is provided by recombinant expression of a nucleic acid encoding a PKCθ polypeptide.

4. The method of claim 3, wherein the nucleic acid comprises a sequence as set forth in SEQ ID NO:1 or a nucleic acid encoding an amino acid sequence as set forth in SEQ ID NO:2.

5. The method of claim 3, wherein the nucleic acid is expressed in vivo.

6. The method of claim 5, wherein the nucleic acid is expressed in a transfected cell.

7. The method of claim 5, wherein the in vivo expression comprises expression of a heterologous PKCθ polypeptide in a non-human transgenic animal.

8. The method of claim 1, wherein the activity of the PKCθ polypeptide is determined by measuring the activity of an IβB-kinase β (IKKβ).

9. The method of claim 1, wherein the activity of the PKCθ polypeptide is determined by measuring the activity of an NFκB.

10. The method of claim 1, wherein the activity of the PKCθ polypeptide is determined using a phosphorylation assay.

11. The method of claim 1, wherein the activity of the PKCθ polypeptide is determined by measuring the activity of a reporter construct comprising an NFκB-responsive element.

12. The method of claim 11, wherein the NFκB-responsive element comprises an HIV-1 promoter.

13. The method of claim 1, wherein the detecting an activity of PKCθ comprises detecting a change in an HIV promoter activity.

14. The method of claim 11, wherein the NFκB-responsive element comprises an IL-2 promoter.

15. The method of claim 11, wherein the NFκB-responsive element comprises a CD28RE/AP-1 element.

16. The method of claim 1, wherein the activity of the PKCθ polypeptide is determined by measuring the activity of a reporter construct comprising an AP-1-responsive element.

17. The method of claim 1, wherein the activity of the PKCθ polypeptide is determined by measuring the activity of a reporter construct comprising an fas ligand promoter.

18. The method of claim 1, wherein the activity of the PKCθ polypeptide is determined by measuring the activity of a reporter construct comprising a serum response element (SRE) promoter.

19. The method of claim 5, wherein a PKCθ polypeptide and a reporter construct are co-expressed.

20. The method of claim 1, wherein the agent inhibits an activity of the PKCθ polypeptide.

21. The method of claim 20, wherein the agent inhibits an NFκB activity.

22. The method of claim 1, wherein determining an activity of the PKCθ comprises detecting activation of NFκB or an NFκB signaling pathway.

23. The method of claim 1, wherein determining an activity of the PKCθ comprises detecting binding of the PKCθ or PKCθ kinase activity.

24. The method of claim 1, wherein determining an activity of the PKCθ polypeptide comprises detecting the ability of the test compound to inhibit the translocation of a PKCθ polypeptide to a cell membrane after co-stimulation of a T cell receptor (TCR) and a CD28.

25. The method of claim 24, wherein the test compound inhibits the translocation of a PKCθ polypeptide to a T cell synapse in the cell membrane.

26. The method of claim 24, wherein the test compound inhibits the translocation of a PKCθ polypeptide to a lipid raft.

27. The method of claim 24, wherein the test compound inhibits the association of a PKCθ polypeptide to a cytoskeletal protein.

28. The method of claim 1, wherein determining an activity of the PKCθ comprises detecting a tyrosine kinase p59fyn activity.

29. A method for identifying a therapeutic agent for ameliorating an HIV infection comprising:
(a) providing a PKCθ polypeptide and a test agent;
(b) contacting the PKCθ polypeptide with the test agent; and,
(c) determining the activity of the PKCθ polypeptide, wherein an increase or decrease in activity of the PKCθ polypeptide in the presence of the test agent thereby identifies the agent as a modulator of PKCθ polypeptide activity and a therapeutic agent for ameliorating an HIV infection.

30. A method for identifying a therapeutic agent for ameliorating an HIV infection comprising:
(a) recombinantly expressing a PKCθ polypeptide in a cell;
(b) contacting the cell with a test agent; and,
(c) determining the activity of the PKCθ polypeptide, wherein an increase or decrease in activity of the PKCθ polypeptide in the presence of the test agent thereby identifies the agent as a modulator of PKCθ polypeptide activity and a therapeutic agent for ameliorating an HIV infection.

31. The method of claim 30, wherein the activity of the PKCθ polypeptide is determined by measuring the activity of a reporter construct.

32. The method of claim 31, wherein the reporter construct comprises an HIV-1 promoter.

33. The method of claim 32, wherein detecting an activity of PKCθ comprises detecting a change in the HIV promoter's activity.

34. A method for ameliorating a condition in a subject, wherein the condition can be ameliorated by modulating PKCθ polypeptide activity, comprising administering to a subject a pharmaceutical formulation comprising an effective amount of an agent capable of modulating a PKCθ polypeptide activity, thereby ameliorating the condition in the subject.

35. A method for ameliorating an HIV infection in a subject, comprising administering to a subject a pharmaceutical formulation comprising an effective amount of an agent capable of modulating a PKCθ polypeptide activity, thereby ameliorating the HIV infection in the subject.

36. A method for ameliorating an immune disorder in a subject, comprising administering to a subject a pharmaceutical formulation comprising an effective amount of an agent capable of modulating a PKCθ polypeptide activity, thereby ameliorating the immune disorder in the subject.

37. The method of claim 36, wherein the immune disorder is a graft versus host disease.

38. The method of claim 36, wherein the immune disorder is an autoimmune disease

39. A method for ameliorating a skeletal muscle disorder in a subject, comprising administering to a subject a pharmaceutical formulation comprising an effective amount of an agent capable of modulating a PKCθ polypeptide activity, thereby ameliorating the skeletal muscle disorder in the subject.

40. A method for ameliorating a condition in a subject, wherein the condition can be ameliorated by modulating PKCθ polypeptide activity, comprising administering to a subject a pharmaceutical formulation comprising an effective amount of an agent capable of modulating a PKCθ polypeptide activity, thereby ameliorating the condition in the subject, wherein the agent is a dominant negative or dominant positive PKCθ polypeptide.

41. A method for ameliorating a condition in a subject, wherein the condition can be ameliorated by modulating PKCθ polypeptide activity, comprising administering to a subject a pharmaceutical formulation comprising an effective amount of an agent capable of modulating a PKCθ polypeptide activity, thereby ameliorating the condition in the subject, wherein the agent comprises a nucleic acid comprising a PKCθ antisense sequence capable of modulating the expression of a PKCθ polypeptide in a cell.

42. A method for ameliorating a condition in a subject, wherein the condition can be ameliorated by modulating PKCθ polypeptide activity, comprising administering to a subject a pharmaceutical formulation comprising an effective amount of an agent capable of modulating a PKCθ polypeptide activity, thereby ameliorating the condition in the subject, wherein the agent comprises a PKCθ-specific antibody, or PKCθ polypeptide-binding fragment thereof.

43. A method for ameliorating a condition in a subject, wherein the condition can be ameliorated by modulating PKCθ polypeptide activity, comprising administering to a subject a pharmaceutical formulation comprising an effective amount of an agent capable of modulating a PKCθ polypeptide activity, thereby ameliorating the condition in the subject, wherein the agent comprises a rottlerin or a functional equivalent thereof.

44. A method for ameliorating a condition in a subject, wherein the condition can be ameliorated by modulating PKCθ polypeptide activity, comprising administering to a subject a pharmaceutical formulation comprising an effective amount of an agent capable of modulating a PKCθ polypeptide activity, thereby ameliorating the condition in the subject, wherein the agent inhibits binding of a lipid cofactor to PKCθ.

45. A method for ameliorating a condition in a subject, wherein the condition can be ameliorated by modulating PKCθ polypeptide activity, comprising administering to a subject a pharmaceutical formulation comprising an effective amount of an agent capable of modulating a PKCθ polypeptide activity, thereby ameliorating the condition in the subject, wherein the agent binds a nucleic acid regulating PKCθ expression.
